(19) United States
(12) Patent Application Publication
(10) Pub. No.: US 2010/0010087 A1
(43) Pub. Date: Jan. 14, 2010

(54) Methods for Inducing Stem Cell Migration and Specialization with EC-18

(76) Inventor: John J. Hong, Flemington, NJ (US)

Correspondence Address: ANTOINETTE G. GIUGLIANO, P.C.;DBA AGG Intellectual Property Law, 100 Cummings Center, Suite 213C, Beverly, MA, 01915, US

(21) Appl. No.: 12/500,961

(22) Filed: Jul. 10, 2009

Related U.S. Application Data
(60) Provisional application No. 61/079,550, filed on Jul. 10, 2008.

(51) Int. Cl. A61K 31/22; A61P 25/00

(52) U.S. Cl. 514/549

(57) ABSTRACT

The present invention relates to promoting stem cell migration and specialization by administering one or more MADG compounds. The present invention further pertains to repairing or promoting the healing of injured tissue or a wound of an individual by administering a MADG compound (e.g., EC-18) to the individual, or subjecting the injured tissue/wound to a MADG compound. Another aspect of the invention relates to treating disease or conditions that benefit from stem cell therapy by administering MADG with or without exogenous stem cells.

### RELATED APPLICATION

This application claims the benefit of U.S. Provisional Application No. 61/079,550, filed Jul. 10, 2008, entitled “Methods for Inducing Stem Cell Migration and Specialization with EC-18”. The entire teachings of the above application are incorporated herein by reference.

### BACKGROUND OF THE INVENTION

The ability to control and guide stem cells to migrate and differentiate into specific types of cells would allow one to treat a wide variety of diseases. Stem cell therapy is promising and has a wide clinical application to a number of diseases, and tissue injury. Conditions in which stem cell therapy is implicated include diabetes, spinal cord injury, brain injury, cardiac injury, and the like. However, stem cell therapy, in many instances, can be refined and improved.

Hence, a need exists to promote the migration and specialization of stem cells. A further need exists to enhance stem cell therapy. Yet a further need exists to improve treatments of spinal cord injury, brain injury, and other diseases that benefit from stem cell therapy.

### SUMMARY OF THE INVENTION

The present invention relates to a method of treating an individual having a wound or injured tissue. The steps of the method encompass selecting an individual having a wound or injured tissue; and administering an amount of one or more mono acetyl diacyl glycerol compounds (referred to herein as “MADG”) to the individual.

One of MADG compounds used in the present invention includes 1-palmitoyl-2-linoleoyl-3-acetyl-rac-glycerol (also referred to herein as EC-18). In another embodiment, the present invention relates to administering a MADG compound or derivative that is represented by the following formula I.

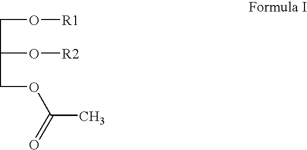

Wherein, R1 is 9-octadecenoyl and R2 is hexadecanoyl; R1 is hexadecanoyl and R2 is 9-octadecenoyl; R1 is hexadecanoyl and R2 is 9,12-octadecadiennoyl; R1 is hexadecanoyl and R2 is 9,12,15-octadecatriennoyl; or R1 is hexadecanoyl and R2 is 5,8,11,14-eicosatetraenoyl. In another embodiment, the MADG compound for use in the methods of the present invention is represented by Formula II:

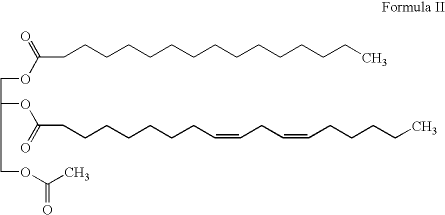

MADG is administered in an amount that ranges between about 10 mg/kg and about 100 mg/kg. The administration of a MADG compound enhances healing of the wound or injured tissue, or alleviates or ameliorates symptoms associated with the wound or injured tissue, as compared to that not subjected to a MADG compound. The injured tissue includes, e.g., myocardial injury, brain injury, spinal cord injury, muscular injury, skeletal injury, or acute tubular necrosis. Symptoms of wounds or injured tissue that are ameliorated include bleeding, inflammation, edema, bruising, and elevation of leukocytes.

Methods of inducing stem cell migration, stem cell specialization, or both (e.g., in an individual) are also included by the methods of the present invention. The steps of the methods involve subjecting tissue to an amount of one or more MADG compounds (in vivo or in vitro); wherein stem cell migration, stem cell specialization, or both are induced in the tissue. The methods can further include a step of assessing stem cell migration, stem cell specialization, or both. In yet another aspect, a MADG compound increases expression of SDF-1. The method further includes, e.g., subjecting the tissue to an amount of stem cells (e.g., stem cell therapy).

Similarly, the present invention also relates to a method of treating an individual with a disease that benefits from stem cell therapy. MADG administration controls stem cell migration and activation, and applies to both endogenous as well as exogenous stem cells. As such, the present invention pertains to MADG administration to individuals with and without the additional administration of stem cells. In the embodiment of endogenous stem cell, the present invention includes administering an amount of one or more MADG glycerol compounds to individuals having a disease or condition. In the aspect of exogenous stem cells, the method includes administering an amount of a MADG compound to the individual; and administering an amount of stem cells to the individual (e.g., in an amount that ranges between about 100,000 cells and about 160 million cells), wherein the individual is treated. Diseases or conditions that are implicated by these methods include those that generally benefit from stem cell therapy or those that involve tissue repair or regeneration. Examples of such disease or conditions include myocardial injury, brain injury, spinal cord injury, muscular injury, skeletal injury, acute tubular necrosis, bowel injury, lung injury, liver injury, kidney injury, bone injury, CNS injury, Alzheimers, Parkinsons, skin injury, hernia repair, vascular anastomoses, after blunt or penetrating traumatic injury, and diabetes.

Advantageously, the present invention allows for improved migration and specialization stem cells, and the promotion of healing of injured tissue or wounds. Additionally, the present invention increases the effectiveness of stem cell therapy, by enhancing stem cell migration and specialization. Finally, the present invention provides for the administration of one or more MADG compounds along with stem cell therapy to provide better and more effective treatments of diseases that benefit from stem cell therapy.

### BRIEF DESCRIPTION OF THE DRAWINGS

FIG. 1 is a photograph of showing the bioluminescence of Aequorea, a hydromedusa.

FIG. 2 is schematic showing the process for a caecal ligation and puncture (CLP).

FIG. 3 a graph showing the survival rate of septic mice over time (hours) after administration of EC-18 simultaneously with CLP, EC-18 at 1 hour after CLP, and PBS (control).

FIG. 4A is a graph showing Cxcl2 (SDF-1) mRNA expression in the lung in mice over time (hours) with and without administration of EC-18.

FIG. 4B is a graph showing kit (SCF) mRNA expression in the lung in mice over time (hours) with and without administration of EC-18.

FIG. 5A is a graph showing Cxcl2 (SDF-1) mRNA expression in the liver in mice over time (hours) with and without administration of EC-18.

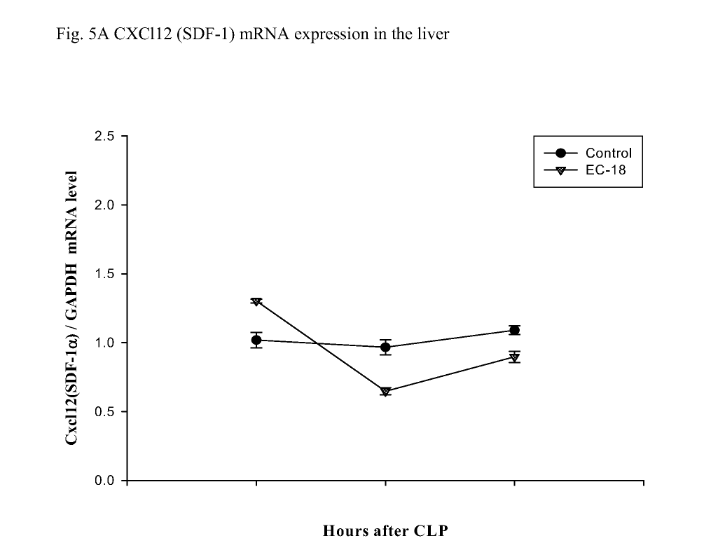

FIG. 5B is a graph showing kit (SCF) mRNA expression in the liver in mice over time (hours) with and without administration of EC-18.

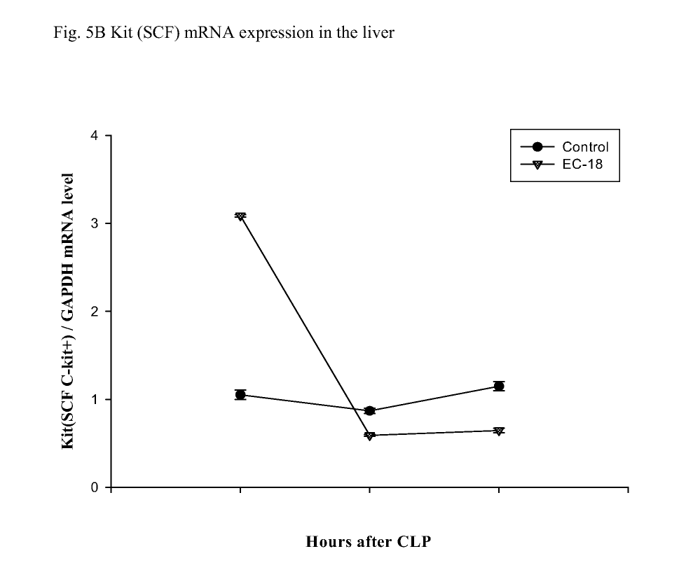

### DETAILED DESCRIPTION OF THE INVENTION

A description of preferred embodiments of the invention follows.

The present invention relates to a new treatment for wounds or injured tissue. In particular, the present invention is based on the unexpected discovery that subjecting tissue or cells to a MADG compound induces stem cell migration and specialization. MADG compounds surprisingly activate, stimulate or otherwise increase production of SDF-1, which induces stem cell migration and specialization. The present invention relates to the administration of one or more MADG compounds for inducing tissue repair and regeneration, and to treat wounds or injured tissue (e.g., from myocardial injury, brain injury, spinal cord injury, skeletal injury, and acute tubular necrosis).

In one aspect, a MADG compound for use with the methods of the present invention include EC-18, also known as 1-palmitoyl-2-linoleoyl-3-acetyl-rac-glycerol. Additionally, the MADG compound for use with the methods of the present invention refers to a compound or derivative that is represented by the following Formula I as an effective ingredient.

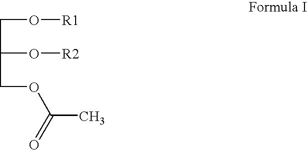

wherein, RI/R2 is 9-octadecenoyl(oleoyl)/hexadecanoyl(palmitoyl), hexadecanoyl(palmitoyl)/(9-octadecenoyl(oleoyl), hexadecanoyl(palmitoyl)/9,12-octadecadienoyl(linoleoyl), hexadecanoyl(palmitoyl)/9,12,15-octadecatrienoyl(linolenoyl) or hexadecanoyl(palmitoyl)/5,8,11,14-eicosatetraenoyl(arachidonoyl). In a particular embodiment, the MADG compound is represented by the Formula II:

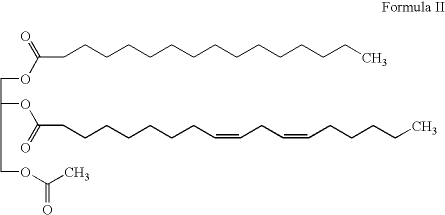

These compounds can be extracted from Cervus nippon (C.N.) antlers using methods known in the art. These compounds can also be chemically synthesized using methods known in the art. See US 2008/0200543 A1. Example 2 describes methods for both extraction and synthesis of MADG compounds of the present invention.

Specifically, the present invention utilizes the following compounds:
Compound 1: 1-oleoyl-2-2palmitoyl-3-acetylglycerol (R1/R2=9-octadecenoyl/hexadecanoyl)
Compound 2: 1-palmitoyl-2-oleoyl-3-acetylglycerol (R1/R2=hexadecanoyl/9-octadecenoyl)
Compound 3: 1-palmitoyl-2-linoleyl-3-acetylglycerol (R1/R2=hexadecanoyl/9,12-octa decadienoyl)
Compound 4: 1-palmitoyl-2-linolenoyl-3-acetylglycerol (R1/R2=hexadecanoyl/9,12,15-octadecatrienoyl)
Compound 5: 1-palmitoyl-2-arachidonoyl-3-acetylglycerol (R1/R2=hexadecanoyl/5,8,11,14-eicosatetraenoyl)
   In an embodiment, any of the MADG compounds represented by formula 2 can be used by the present invention, or MADG compounds, Compounds 1-5 represented above. In a preferred embodiment, Compound 3 is used with the methods of the present invention.

In the course of the experiments done with MADG compounds (e.g., EC-18), it was determined that MADG compounds stimulated increases in expression of mRNA coding for a protein, stromal cell-derived factor (SDF-1). In an embodiment, the beneficial biological effects of MADG compounds are related to these increases in SDF-1 production by the bone marrow, and in the lung and liver in the abdominal infection animal model. SDF-1 is also intimately related to migration and specialization of endogenous stem cells from the bone marrow to sites of injury and tissue repair. MADG compounds stimulate the secretion of SDF-1 at sites of tissue damage or organ injury, and is therefore beneficial in the treatment of a wide variety of diseases, including disease that benefit from exogenous embryonic stem cell therapy.

Since MADG compounds lead to increases in SDF-1, they can be used in the clinical treatment of a wide variety of diseases, comparable, in many ways to the use of exogenous embryonic stem cells. Treatment with a (e.g., one or more) MADG compound can be used alone or together with exogenous embryonic stem cell therapy. Furthermore, a MADG compound can be used as a complement the use of exogenous stem cells as well, by improving “homing” and specialization of those cells to sites of tissue injury and repair. Since MADG compounds are involved in the control of stem cell migration and activation, the present invention relates to administering one or more MADG compounds with and without stem cell therapy. In the case of endogenous stem cells, those stem cells already existing in the individual, administration of a MADG compound causes these stem cells to migrate and specialize to the area of injury. MADG compounds (e.g., EC-18) recruit stem cells from bone marrow and allow them to migrate to specific sites of tissue injury. As such, the administration of stem cell therapy, in this embodiment, is not needed. However, in the case of exogenous stem cell, the present invention involves administration of a MADG compound along with stem cells. As further described herein, administration of MADG aids in the treatment of diseases or conditions that would otherwise benefit from stem cell therapy. Such diseases and conditions are described herein.

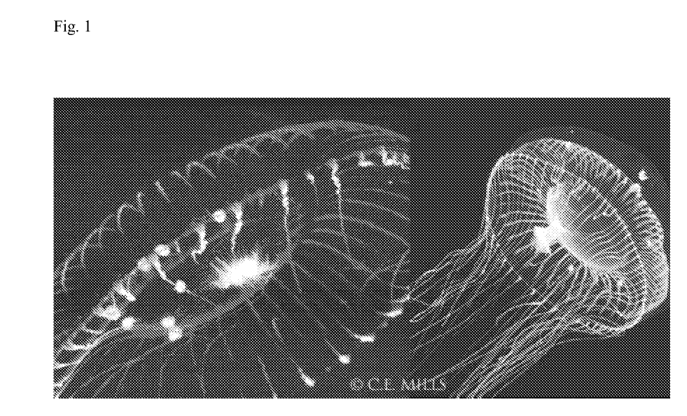

Referring to FIG. 1, Green Fluorescent Protein (GFP) is responsible for the bioluminescence in the jellyfish Aequorea victoria. GFP has been used as a simple marker in vivo and in vitro. An enhanced GFP transgenic mouse was produced, and all its cells except erythrocytes and hair cells were GFP+. These GFP transgenic mice can be used as donors of GFP-labeled bone marrow cells to normal wild-type recipients. The resultant GFP-chimera mice can then be used to study the behavior of bone marrow stem cells (BMSC) in vivo under a wide variety of experimental conditions. GFP chimera mice have been used to investigate BMSC in cutaneous wound healing, and following injury to bowel, lung, liver, kidney, bone, myocardium and CNS.

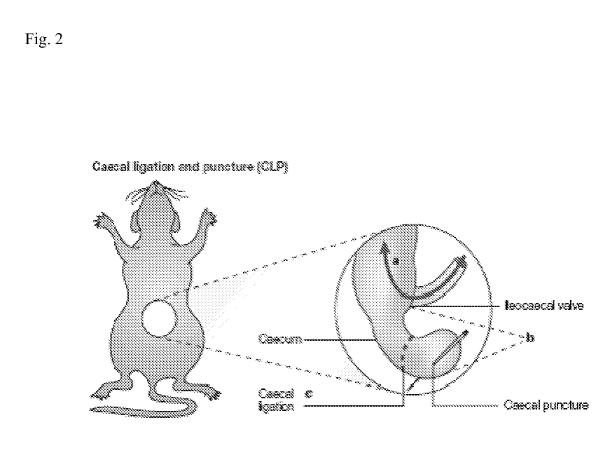

Cecal ligation and puncture (CLP) have been a well-described, standard experimental model of abdominal sepsis (FIG. 2.). Performing CLP on a GFP chimeric mouse allows one to observe the behavior of BMSC in the setting of abdominal sepsis.

The first experiment describes the behavior of BMSC following CLP: FACS, flow cytometry and immunohistochemistry can be used to track the migration of BMSC into peripheral blood, lung, liver, the cutaneous wound, and the primary site of injury following CLP. BMSC behavior can be correlated to time of injury as well as to local (using RT-PCR) and systemic levels of cytokines and chemokines. This experiment can help elucidate the contribution of BMSC to the innate immune response to injury and to local and distant organ and tissue repair and regeneration following CLP.

Following the first set of descriptive experiments, the GFP-mouse chimera model can be especially useful in examining the effects of clinical agents that can enhance BMSC mobilization from the bone marrow niche in the setting of abdominal sepsis.

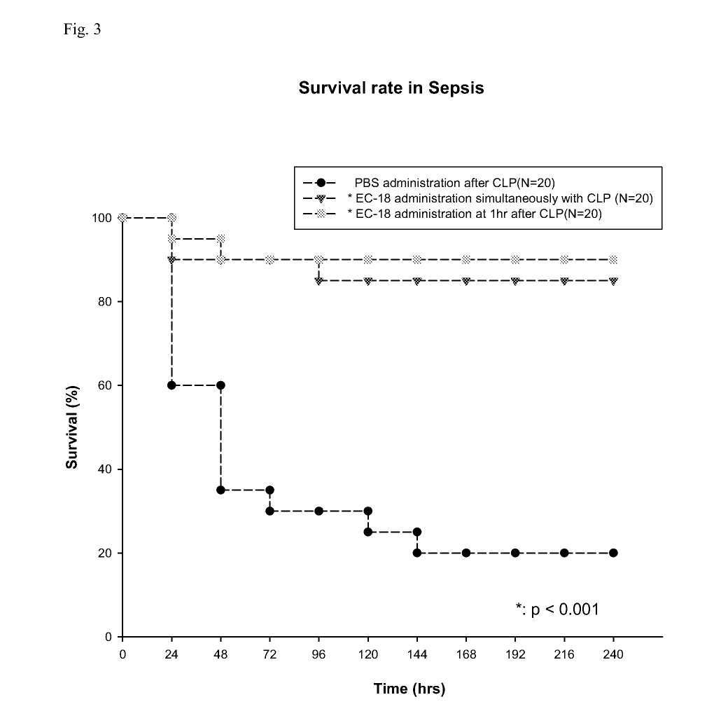

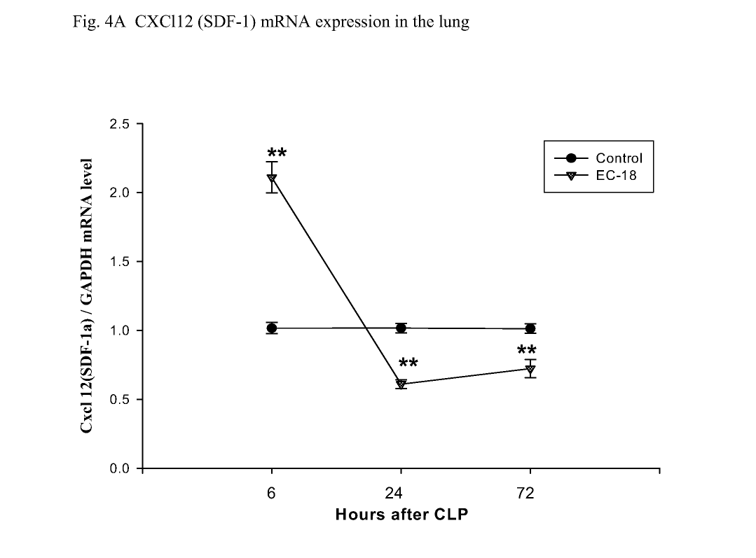

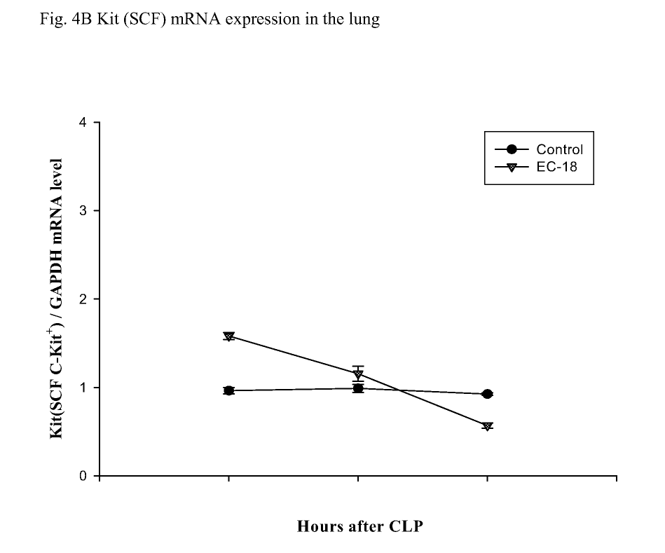

Data with the drug EC-18 have demonstrated a dramatic improvement in survival in mice associated with administration to EC-18 following CLP (FIG. 3.) EC-18 was administered at 50 mg/kg. This improvement in survival is associated with increased early expression of mRNA coding for stromal cell-derived factor 1 (SDF-1) and stems cell factor (SCF) in the lung and the liver as compared to controls. (FIGS. 4A,4B and 5A,5B) These are two chemokines that have been shown to have important roles in BMSC and migration and activation. SDF-1, particularly, has been shown to be an important chemokine affecting BMSC behavior.

SDF-1 is constitutively expressed by bone marrow stromal cells, and presumably maintains immature hematopoietic and stem cells quiescent in the endosteal bone marrow niche. Following tissue injury, SDF-1 expression is increased in the injured peripheral tissues, and this new SDF-1 “gradient” stimulates BMSC migration from the bone marrow into the periphery, aiding in tissue repair and regeneration following injury. Furthermore, SDF-1 has also been shown to play an important role in innate immunity, by potentially modulating local and systemic responses to endotoxin through its interaction with its sole receptor, chemokine receptor 4 (CXCR4), and its effects on Toll-like receptor 4 (TLR4). Briefly, it appears that SDF-1/CXCR4 interact with TLR4 to raise the threshold levels of endotoxin required to stimulate downstream effects of the inflammatory response. Given this dual importance of SDF-1 in sepsis, the beneficial effect of an agent such as MADG compound, which increases local SDF-1 levels in peripheral tissues, is seen.

Finally, the GFP chimera mouse model lends itself to investigate the in vivo effects of MADG beyond the abdominal sepsis model of CLP, applied to a wide variety of organ injury and regeneration models as noted herein. Since BMSC are involved in tissue recovery and regeneration following injury, clinical agents that affect stem cell migration and specialization, such as EC-18, can have clinical utility in a wide variety of diseases. The GFP-mouse chimera model allows for direct testing of this.

For example, like the bone marrow, the skin represents a population of cells that have rapid turnover and ongoing regenerative properties, with the additional benefit of better accessibility than bone marrow. Wound healing experiments using the GFP-mouse chimera model have been performed; experiments investigating the effects of MADG administration on wound healing have never been reported in the literature, and represent an important step. Similar experiments can be done using this mouse model, and can have clinical implications for the use of MADG in the setting of myocardial injury, brain injury, spinal cord injury, skeletal injury, acute tubular necrosis, etc.

The present invention pertains to methods for preventing or treating an individual having a wound or injured tissue. Unexpectedly, MADG, and particularly EC-18, induces tissue repair and regeneration. To treat an individual with a wound or injured tissue means to assist with, improve or increase the rate of repair and regeneration of the injured tissue/wound, as compared to the rate of repair and regeneration of injured tissue/would with no treatment. In an embodiment, to treat an individual with a wound or injured tissue also means to alleviate, ameliorate or reduce the severity of one or more of its symptoms associated with the wound or injured tissue, as compared to a control. In yet another embodiment, the present invention involves increasing stem cell migration and specialization at the wound site or injured tissue site by subjecting the individual or the wound/injured tissue to one or more MADG compounds. Exposure to a MADG compound increases stem cell migration and specialization, as compared to the same conditions without a MADG compound exposure. A MADG compound, can be co-administered with another compound, e.g., another MADG compound or with stem cell therapy, or a compound to target the site of injury.

The wound or injured tissue encompassed by the present invention pertains to, in an embodiment, wounds occurring from a trauma, and includes open and closed wounds, cuts, lacerations, abrasions, incisions, penetration wounds, punctures, contusions, hematomas, blunt force wounds, and the like. Injured tissue includes various injuries such as spinal cord injury, brain injury, muscular injury, myocardial injury, skeletal injury, and abdominal injury. Injury can occur from trauma or surgery, but also from disease, or thrombotic events. For example, brain injury can occur from a stroke and myocardial injury can occur from cardiac arrest, both of which are further described herein.

Symptoms of wounds or tissue injury as a result of trauma include bleeding, inflammation, edema, bruising, elevation of leukocytes. The present invention also relates to methods for reducing severity of the wound or the tissue injury, or by promoting or assisting in tissue regeneration or repair. Reducing the severity of the wound or the tissue injury refers to a reduction in the degree of at least one symptom of the wound or tissue injury (e.g., reducing bleeding, bruising, inflammation), as compared to symptoms without exposure to a MADG compound (e.g., EC-18). Assisting in or promoting tissue repair refers to decreasing the amount of time a wound takes to heal, and/or increasing tissue regeneration, as compared to the amount of time healing or tissue regeneration would need for a similar wound without exposure to a MADG compound. Tissue regeneration refers to cells that migrate to the wound area and allow for the replacement and/or repair of the wounded cell type, or cells that support healing of the wounded cell type. The present invention relates to methods of treating wounds or injured tissue, or reducing symptoms associated therewith by administering to an individual an effective amount of a MADG compound.

Tissue injury can occur from trauma or from a disease or condition that causes tissue injury. In the case of thrombotic events, e.g., cardiovascular and/or cerebrovascular events, include an ischemic stage and a necrotic stage. A patient can suffer from ischemia in which a decrease of blood flow can occur. This decrease in blood flow causes a decrease in tissue oxygenation. After prolonged ischemia, the tissue can undergo necrosis which is death of the tissue. Ischemic and necrotic tissue can be treated with a MADG compound. Patients who undergo a vascular event can exhibit elevated levels of ischemic markers and/or necrosis markers. In an embodiment, treatment with MADG will reduce markers associated with the disease or condition, or prevent these markers from increasing.

As described above, an embodiment of the invention includes treating individuals who have myocardial injury or brain injury by administering MADG. Symptoms that are indicative of a myocardial injury include a history of chest pain, abnormal electrocardiograms, elevated levels of ischemic markers, necrosis markers, or thrombin/fibrin generation markers. Such markers include, but are not limited to, Creatine Kinase with Muscle and/or Brain subunits (CKMB), D-Dimer, F1.2, thrombin anti-thrombin (TAT), soluble fibrin monomer (SFM), fibrin peptide A (FPA), myoglobin, thrombin precursor protein (TPP), platelet monocyte aggregate (PMA) and troponin.

Myocardial injury can be cause from cardiovascular diseases such as coronary heart disease, myocardial infarction, angina or a disease in which a narrowing of a blood vessel occurs in at least one major artery. Brain injury can be the result of cerebrovascular diseases including stroke or transient ischemic attacks. Brain injury can also be the result of direct brain trauma from a head injury; or from degenerative brain conditions such as Alzheimers and Parkinsons. Other types of injuries can result from deep vein thrombosis or peripheral vascular thrombosis.

Applicable tissue markers can vary depending on the tissue that has been injured or is in need of repair. In the setting of myocardial infarction, as described above, CPK and troponin are routinely used serum markers to detect the presence and severity of cardiac injury. Markers for other types of tissue injury are generally known in the art and can be measured before and after treatment with MADG to determine the status of the tissue injury. After administration of MADG, the injured tissue begins to heal and the marker levels can be assessed to determine the extent of healing.

Another type of injury encompassed by the present invention includes spinal cord injury. Symptoms of spinal cord injury include paralysis, weakness, hypotonia, hyporeflexia, pain, and atrophy of muscles. Administering MADG causes migration and specialization of stem cells, and regeneration of neuronal cells at the injury site. Administration of MADG also reduces the severity of these symptoms. Administration of MADG can be done in conjunction with stem cell therapy, as described in US Publication No. 20090169527 “Materials from Bone Marrow Stromal Cells for Use in Forming Blood Vessels and Producing Angiogenic and Trophic Factors”.

The methods of the present invention are also encompassed by diseases or conditions which require or are treated with stem cell therapy (e.g., administration of stem cells). In particular, the present invention involves administering MADG along with stem cell therapy to enhance stem cell migration and/or specialization. Specifically, MADG is administered with the administration of human embryonic stem cells at the site of injury. The amount of stem cells to be administered will depend on the disease being treated, the tissue being treated and/or the type of injury being treated. In an embodiment, the amount of embryonic stem cells administered ranges from 100,000 cells to 160 million cells.

Methods and preparation for carrying out stem cell therapy are known in the art. For example, in order to prepare the compositions, about 750,000 to about 160 million Human embryonic stem (hES) cells and/or one or more of their derivatives such as hematopoietic stem cell progenitors, neuronal stem cell progenitors, mesenchymal stem cell progenitors, insulin producing stem cell progenitors, hepatocyte stem cell progenitors, cardiac stem cell progenitors, epithelial stem cell progenitors or mixtures thereof are suspended in about 0.25 ml to about 100 ml of a carrier vehicle. In one embodiment, about 750,000 to about 80 million hES cells are suspended in about 0.25 ml to about 10 ml of the carrier vehicle. Enrichment for specific differentiated stem cell progenitor types in a population is a priority, although a proportion of undifferentiated stem cells will remain in the composition. In one embodiment, the portion of undifferentiated stem cells will be no more than about 80% of the total population of cells. In another embodiment, the portion of undifferentiated stem cells will be no more than about 40% of the total population of cells. See US Patent Publication 20090123433 for an example of methods and preparations of stem cell therapy.

Disease or injury that benefit from stem cell therapy or benefit from tissue repair or regeneration are implicated by the methods of the present invention. For example, such diseases or conditions include, e.g., myocardial injury (e.g., from myocardial infarction or angina), brain injury (e.g., from stroke), spinal cord injury, muscular injury, skeletal injury, acute tubular necrosis, bowel injury, lung injury, liver injury, kidney injury, bone injury, CNS injury, Alzheimers, Parkinsons, skin injury, hernia repair, vascular anastomoses, after blunt or penetrating traumatic injury, and diabetes. Additional disease that benefit from stem cell therapy include, for example, liver and kidney disorders, nervous system disorders, autoimmune disorders, genetic disorders, eye disorders, musculoskeletal disorders, fertility and reproductive disorders and cardiovascular disorders and without limitation include Arthritis, Astrocytoma, Auditory Nerve Atrophy, Autism, Auto Immune Disorders, Alzheimer's disease, Ankylosing Spondylitis, Becker's Muscular Dystrophy, Brain Damage, Burns, Cerebrovascular Insult, Cerebral Palsy, Coma, Corneal Ulcers, Comeal Graft Rejection, Cortico-Basal Degeneration of the Nervous System, Coronary Artery Disease, Diabetes, Dementia, Downs Syndrome, Duchenne's Muscular Dystrophy, End-Stage Renal Disease, Erb's Palsy, Fascio Scapular Muscular Dystrophy, Fertility Disorders, Friedereich's Ataxia, Heart Failure, Hepatocellular Carcinoma, Hereditary Spino Motor Neuron Disease, Huntington's Chorea, Krabbe's Disease, Limb Girdle Dystrophy, Liver Cirrhosis, Macular Degeneration, Mental Retardation, Multiple Sclerosis, Motor Neuron Disease, Myocardial Infarction, Nephrotic Syndrome, Niemann Pick Disease, Non-Healing Ulceration of the Skin, Olivo-Ponto Cerebellar Atrophy, Optic Nerve Atrophy, Parkinson's Disease, Post Electric Shock Encephalopathy, Post-Rabies Vaccine Encephalopathy, Pressure Sores, Progressive Supranuclear Palsy, Psoriasis, Pthysis Bulbi, Restrictive Cardiomyopathy, Retinitis Pigmentosa, Right Bundle Branch Block, Sarcoidosis, Sinus Bradycardia, Spinal Muscular Dystrophy, Spino Cerebellar Ataxia, Steven Johnson's Syndrome, Systemic Lupus Erythematosus, Thrombocytopenia, Thalassemia, Ulcerative Colitis, Vegetative State, Cystic Fibrosis, Interstitial Lung Disease, Azoospermia, Primary Ovarian Failure, Aphthous Ulcers, Hormone Imbalance, Osteo-Arthritis, Homer's Syndrome and Osteogenic Imperfecta, along with Channelopathy and Hypogammaglobulinemia. Any disease or condition, now known or later discovered, that is determined to benefit from stem cell therapy can be enhanced by administering MADG. Any disease or condition that involves tissue regeneration or repair can be treated with the methods described herein. MADG provides a noninvasive, nontoxic substance that enhances tissue regeneration and repair throughout the body.

One aspect of the invention, as described herein, includes administering MADG along with at least one other compound or composition that is used for treating the wounds or injured tissue (a “wound treating compound”). For an individual with a wound, MADG can be administered together with stem cell therapy, as further described herein.

In particular, stem cell migration and specialization refers to stem cells that go to the site of injury and specialize into cell types of that which was injured, e.g., to promote tissue repair, or specialize into cell types that support tissue repair. Any type of stem cells, now known or later discovered or engineered, can be used with embodiments of the present invention. Bone marrow, for example, contains three types of stem cells. Hematopoietic stem cells (HSC) give rise to the three classes of blood cells that are found in the circulation: white blood cells (leukocytes), red blood cells (erythrocytes), and platelets (thrombocytes). Mesenchymal stem cells are found around the central sinus in the bone marrow. They have the capability to differentiate into osteoblasts, chondrocytes, myocytes, and many other types of cells. They also function as “gatekeeper” cells of the bone marrow. Endothelial stem cells that differentiate into skin cells. Also encompassed by the present invention is embryonic stem cell therapy and administration.

Stem cells are cells can turn into multiple cell types (multipotency) and/or have the ability to self-renew. For example, a small number of HSCs can expand to generate a large number of progeny HSCs. This phenomenon is used in bone marrow transplant when a small number of HSCs reconstitute the hematopoietic system. HSCs have a higher potential than other immature blood cells to pass the bone marrow barrier, and settle elsewhere in the body, and differentiate into cells common to that area (e.g.,in the thymus, liver, spleen, etc.).

To determine if MADG (e.g., EC-18) increases migration and specialization of stem cells to a site of injury, certain markers can be assessed or measured. For example, a sample (e.g., a tissue sample) can be taken and the markers can be assessed. HSC are identified by their small size, lack of lineage markers, low staining with vital dyes such as rhodamine 123 or Hoechst 33342, and presence of various antigenic markers on their surface, many of which belong to the cluster of differentiation series, like: CD34, CD38, CD90, CD133, CD105, CD45 and also c-kit—the receptor for stem cell factor. The HSC are generally negative for the markers that are used for detection of lineage commitment, and are, thus, called Lin-; and, during their purification by FACS, a number of different mature blood-lineage markers can be assayed, e.g., CD13 & CD33 for myeloid, CD71 for erythroid, CD19 for B cells, CD61 for megakaryocytic, etc. for humans; and, B220 (murine CD45) for B cells, Mac-1 (CD11b/CD18) for monocytes, Gr-1 for Granulocytes, Ter119 for erythroid cells, I17Ra, CD3, CD4, CD5, CD8). Hence, the present invention relates to administration of MADG (e.g., EC-18) to an individual having tissue injury, and assessing the extent of stem cell migration and/or specialization, which can be measured by markers, as described herein. This aspect can also be demonstrated using the GFP mouse model, as described herein, because the GFP chimera mouse have fluorescent labeled bone marrow, which is easily traced around the body within different tissue depending on the nature of the injury being studied.

In general, the wound healing process involves about three phases: inflammation phase, the proliferative phase and the remodeling phase. In the inflammatory phase, clotting takes place in order to obtain hemostasis, or stop blood loss, and various factors are released to attract cells that phagocytise debris, bacteria, and damaged tissue and release factors that initiate the proliferative phase of wound healing. In the proliferative phase, fibroblasts begin to enter the wound site and angiogenesis, the formation of new blood vessels occurs, resulting in tissue regeneration and repair. The remodeling phase is a phase in which collagen is remodeled and realigned. These phases can overlap, and MADG administration or exposure can occur at any time during any one of these phases to facilitate stem cell migration and specialization. In an embodiment, MADG administration occurs close in time from when the individual receives the wound or tissue injury. In a preferred embodiment, MADG administration occurs during the inflammation phase.

The present invention relates to co-administering MADG along with HSC or e.g., embryonic stem cells for treatment of the diseases described herein. MADG can be administered before, or after HSC/embryonic stem cells so long as they are administered close enough in time to confer the desired effect of promoting stem cell migration and specialization.

In the case of diabetes, type I can be treated with stem cells to induce proliferation of insulin secreting cells. For example, stem cells can be differentiated into pancreatic beta cells that produce insulin. As such, the present invention relates to methods for treating type I diabetes by administering MADG, along with stem cells that differentiate into insulin producing cells.

Similarly, stroke and traumatic brain injury lead to cell death, characterized by a loss of neurons and oligodendrocytes within the brain. Healthy adult brains contain neural stem cells, these divide and act to maintain general stem cell numbers or become progenitor cells. Stem cells can be administered with MADG, as described herein to facilitate differentiation of neuronal cells.

Spinal cord injury is another condition in which the methods of the present invention can be used. Stem cells, along with MADG can be administered, e.g., injected at the site of the spinal cord injury to induce growth of neuronal cells and cells of the myelin sheath for repair of the spinal cord.

As with brain and spinal cord injury, heart damage and any type of injury described herein, can also be repaired using stem cell therapy co-administered with MADG.

### Modes and Manner of Administration, Dosages

MADG, as used with the methods of the present invention, can be administered with or without a carrier. The terms “pharmaceutically acceptable carrier” or a “carrier” refer to any generally acceptable excipient or drug delivery composition that is relatively inert and non-toxic. Exemplary carriers include sterile water, salt solutions (such as Ringer's solution), alcohols, gelatin, talc, viscous paraffin, fatty acid esters, hydroxymethylcellulose, polyvinyl pyrolidone, calcium carbonate, carbohydrates (such as lactose, sucrose, dextrose, mannose, albumin, starch, cellulose, silica gel, polyethylene glycol (PEG), dried skim milk, rice flour, magnesium stearate, and the like. Suitable formulations and additional carriers are described in Remington's Pharmaceutical Sciences, (17^{th} Ed., Mack Pub. Co., Easton, Pa.). Such preparations can be sterilized and, if desired, mixed with auxiliary agents, e.g., lubricants, preservatives, stabilizers, wetting agents, emulsifiers, salts for influencing osmotic pressure, buffers, coloring, preservatives and/or aromatic substances and the like which do not deleteriously react with the active compounds. Typical preservatives can include, potassium sorbate, sodium metabisulfite, methyl paraben, propyl paraben, thimerosal, etc. The compositions can also be combined where desired with other active substances, e.g., enzyme inhibitors, to reduce metabolic degradation. A carrier (e.g., a pharmaceutically acceptable carrier) is preferred, but not necessary to administer the compound.

One or more MADG compounds can be a liquid solution, suspension, emulsion, tablet, pill, capsule, sustained release formulation, or powder. The method of administration can dictate how the composition will be formulated. For example, the composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc.

One or more MADG compounds can be administered intravenously, parenterally, intramuscular, subcutaneously, orally, nasally, topically, by inhalation, by implant, by injection, or by suppository. The composition can be administered in a single dose or in more than one dose over a period of time to confer the desired effect.

The actual effective amounts of compound or drug can vary according to the specific composition being utilized, the mode of administration and the age, weight and condition of the patient. For example, as used herein, an effective amount of the drug is an amount which induces tissue regeneration or repair, or which promotes stem cell migration and/or specialization. Dosages for a particular individual patient can be determined by one of ordinary skill in the art using conventional considerations, (e.g. by means of an appropriate, conventional pharmacological protocol). In one embodiment, a MADG compound (e.g., EC-18) can be administered in an amount between about 10 mg/kg and about 100 mg/kg once, or periodically (e.g., more than once a day, daily, weekly, monthly) over the course of treatment. In a preferred embodiment, a MADG compound is administered in a range between about 40 mg/kg and about 80 mg/kg, and in an aspect, at about 50 mg/kg.

In one embodiment, the composition for use with the present invention comprises at least about 20% to about 100% by weight of one or more MADG compounds. In yet another embodiment, the MADG compound used with the methods of the present invention include about 60% to about 80% by weight. For properties of MADG compounds, see Yang HO, Kim SH et al. Chem Pharm Bull 52(7)874-78;2004, the teachings of which are incorporated herein by reference.

For enteral or mucosal application (including via oral and nasal mucosa), particularly suitable are tablets, liquids, drops, suppositories or capsules. A syrup, elixir or the like can be used wherein a sweetened vehicle is employed. Liposomes, microspheres, and microcapsules are available and can be used.

Pulmonary administration can be accomplished, for example, using any of various delivery devices known in the art such as an inhaler. See. e.g., S. P. Newman (1984) in Aerosols and the Lung, Clarke and Davis (eds.), Butterworths, London, England, pp. 197-224; PCT Publication No. WO 92/16192; PCT Publication No. WO 91/08760.

For parenteral application, particularly suitable are injectable, sterile solutions, preferably oily or aqueous solutions, as well as suspensions, emulsions, or implants, including suppositories. In particular, carriers for parenteral administration include aqueous solutions of dextrose, saline, pure water, ethanol, glycerol, propylene glycol, peanut oil, sesame oil, polyoxyethylene-polyoxypropylene block polymers, and the like. Ampules are convenient unit dosages.

The administration of MADG along with other forms of treatment can occur simultaneously or sequentially in time. Other forms of treatment include administration of stem cells (e.g., stem cell therapy) or other compound (e.g., wound healing compound) and can be administered before, after or at the same time as MADG. Thus, the term “co-administration” is used herein to mean that MADG and other forms of treatment will be administered at times to achieve a treatment of the disease, tissue repair, stem cell migration, stem cell specialization or combination thereof. The methods of the present invention are not limited to the sequence in which MADG and an addition form of treatment are administered, so long as they are administered close enough in time to produce the desired effect of repairing tissue, inducing stem cell migration and/or specialization, or treating the disease in question.

### Exemplification

### EXAMPLE 1

### A Study of the in Vivo Behavior of Endogenous Bone Marrow Stem Cells in the Setting of Abdominal Sepsis

Using a well-established animal model, the effects of EC-18 on endogenous bone marrow stem cells can be demonstrated. The initial experiment involves EC-18's effect in the setting of intraabdominal infection. This experiment demonstrates that EC-18 affects mobilization and specialization of stem cells of bone marrow origin to local and remote sites of injury.

### Methods

Specifically, donor transgenic GFP (C57BL/6) mice are given 5-FU (150 mg/kg) to kill mature cells. Bone marrow is harvested. C57BL/6 congenic recipient mice are exposed to lethal doses of whole-body radiation (10Gy Cesium-137 at 1.0 Gy/min) and then undergo bone marrow transplant. Engraftment of GFP-BMSC is confirmed after five weeks in select sacrificed mice. Surviving mice are GFP-BMSC chimeras available for investigation. These GFP-BMSC mice then undergo CLP. Specifically, the mice are anesthetized and undergo laparotomy. The sham group undergo laparotomy only. The non-sham group has the cecum ligated and punctured (size and number of punctures may vary depending on the desired severity of injury). Mice are sacrificed at six, twelve and 24 hours. Beyond overall assessment of survival, peripheral blood, lung, liver, the surgical wound site and the cecum are harvested and are available for immunohistochemistry, FACS and flow cytometry analysis to evaluate the migration and homing of GFP-BMSC. Serum is analyzed for levels of cytokines (TNF-α, IL-1, IL-2, IL-4, IL-6, IL-10, and IFN-γ) and chemokines (SDF-1 and SCF). Lung, liver and cecum are assayed using RT-PCR for mRNA coding for SDF-1 (Cxcl12) and SCF (Kit) as well as GAPDH. EC-18 was administered at 50 mg/kg.

Additionally, four groups of 20 mice: sham group underwent celiotomy but not CLP; control group underwent CLP and administration of PBS; simultaneous treatment group (STG) had administration of 50 mg/kg MADG PO. Immediately prior to CLP and at 24, 48, and 72 h post CLP, post treatment group (PTG) had initial administration of MADG at 1 h post CLP, and at 24, 48, and 72 h post-op. Survival was followed to 10 days postop. Serum and tissue levels of pro- and anti-inflammatory cytokines were measured. Serum levels of chemokines stromal cell-derived factor (SDF-1) and stem cell factor (SCF) were measured to ascertain if effects of MADG involve stimulation of bone marrow stromal and stem cells. PCR was used to measure SDF and SCF mRNA expression in liver and lung.

Results: PO administration of MADG significantly improved survival in mice following CLP with associated systemic alterations of a variety of cytokines. Increased levels of mRNA coding for SCF and SDF in lung and liver were found following CLP.
Conclusions: PO administration of MADG following CLP results in marked improvement in survival. Cytokine level changes demonstrate associated immunomodulatory effects. These effects are mediated by bone marrow stromal and stem cell activation, evidenced by increases in SDF and SCF.

### EXAMPLE 2

### Extracting or Synthesizing a Mono Acetyl Diacyl Glycerol Compound

The compounds of the present invention were extracted from Cervus nippon (C.N.) antlers or manufactured by conventional organic synthesis method. The exemplary of extraction method has the following steps. Particularly, the chloroform extracts of C.N. antler are obtained by extracting C.N. antler with hexane first and further extracting the residue of the hexane extract with chloroform. The amounts of hexane and chloroform used in this extraction process are enough amounts to impregnate deer antler. Generally, hexane and chloroform are used in the ratio of 4-51 to 1 kg of C.N. antler. The chloroform extract of C.N. antler obtained from such extraction processes, is fractionated and purified by a series of silica gel column chromatography and TLC (Thin layer chromatography). An eluent of the subsequent extracting steps is selected from chloroform/methanol, hexane/ethylacetate.

In order to synthesize a mono acetyl diacyl glycerol compound chemically, for instance, 1-palmitoylglycerine is separated from the products in the reaction of both glycerol and palmitic acid. The objecting mono acetyl diacyl glycerol can be synthesized as esterifying 1-palmitoylglycerine with carboxylic acid compounds such as acetic acid and linoleic acid, and purified as occasion demands. Another method for synthesizing mono acetyl diacyl glycerol derivatives is the acetolysis of phosphatidyl choline.

The relevant teachings of all the references, patents and/or patent applications cited herein are incorporated herein by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method of treating an individual having a wound or injured tissue, the method comprises:
a. selecting an individual having a wound or injured tissue; and
b. administering to the individual an amount of one or more MADG compounds, wherein the amount ranges between about 10 mg/kg and about 100 mg/kg;
wherein the healing of the wound or injured tissue is enhanced, or symptoms associated with the wound or injured tissue are ameliorated, as compared to a wound or injured tissue not subjected to the MADG compound.

2. The method of claim 1, wherein the MADG compound is represented by formula I: 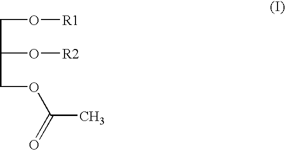
Wherein,
R1 is 9-octadecenoyl and R2 is hexadecanoyl;
R1 is hexadecanoyl and R2 is 9-octadecenoyl;
R1 is hexadecanoyl and R2 is 9,12-octadecadiennoyl;
R1 is hexadecanoyl and R2 is 9,12,15-octadecatriennoyl; or
R1 is hexadecanoyl and R2 is 5,8,11,14-eicosatetraenoyl.

3. The method of claim 1, wherein the MADG compound is represented by formula II:

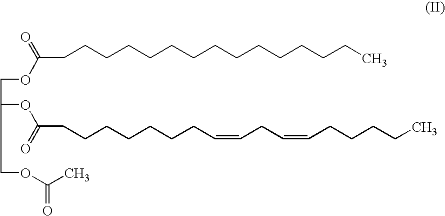

4. The method of claim 1, wherein the injured tissue is the result of acute or chronic diseases or conditions.

5. The method of claim 3, wherein diseases or conditions comprise myocardial injury, brain injury, spinal cord injury, muscular injury, skeletal injury, acute tubular necrosis, bowel injury, lung injury, liver injury, kidney injury, bone injury, CNS injury, Alzheimers, Parkinsons, skin injury, hernia repair, vascular anastomoses, after blunt or penetrating traumatic injury, and diabetes.

6. The method of claim 1, wherein symptoms associated with wound or injured tissue include bleeding, inflammation, edema, bruising, and elevation of leukocytes.

7. A method of inducing stem cell migration, stem cell specialization, or both in tissue, the method comprises:
a. selecting an individual having tissue in need of stem cell migration, stem cell specialization, or both;
b. subjecting the tissue to an amount of one or more MADG compounds, wherein the amount ranges between about 10 mg/kg and about 100 mg/kg;
c. assessing stem cell migration, stem cell specialization, or both;
wherein stem cell migration, stem cell specialization, or both is induced in the tissue; and wherein the MADG compound is represented by formula I: 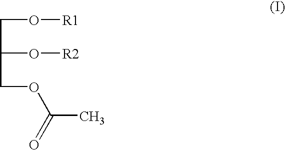
Wherein,
R1 is 9-octadecenoyl and R2 is hexadecanoyl;
R1 is hexadecanoyl and R2 is 9-octadecenoyl;
R1 is hexadecanoyl and R2 is 9,12-octadecadiennoyl;
R1 is hexadecanoyl and R2 is 9,12,15-octadecatriennoyl; or
R1 is hexadecanoyl and R2 is 5,8,11,14-eicosatetraenoyl.

8. The method of claim 7, wherein one or more MADG compounds increases expression of SDF-1.

9. The method of claim 8, further including subjecting the tissue to an amount of exogenous stem cells.

10. A method of promoting tissue repair or regeneration in an individual; the method comprises:
a. selecting an individual in need of tissue repair or regeneration; and
b. administering to the individual an amount of EC-18, wherein the amount ranges between about 10 mg/kg and about 100 mg/kg;
wherein tissue repair or regeneration is promoted, as compared to tissue not subjected to EC-18.

11. A method of inducing stem cell migration, stem cell specialization, or both in an individual having a wound or injured tissue; the method comprises:
a. subjecting the wound or injured tissue to an amount of EC-18, wherein the amount ranges between about 10 mg/kg and about 100 mg/kg;
wherein stem cell migration, stem cell specialization, or both occurs at the wound or injured tissue.

12. The method of claim 11, further including administering an amount of stem cells, wherein the amount of stem cells ranges between about 100,000 cells and about 160 million cells.

13. The method of claim 11, wherein the method further comprises assessing stem cell migration, stem cell specialization or both.

14. A method of treating an individual with a disease that benefits from stem cell therapy, the method comprises:
administering an amount of one or more MADG compounds to the individual, wherein the amount ranges between about 10 mg/kg and about 100 mg/kg, wherein the MADG compound is represented by formula I: 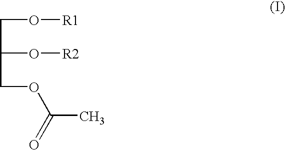
Wherein,
R1 is 9-octadecenoyl and R2 is hexadecanoyl;
R1 is hexadecanoyl and R2 is 9-octadecenoyl;
R1 is hexadecanoyl and R2 is 9,12-octadecadiennoyl;
R1 is hexadecanoyl and R2 is 9,12,15-octadecatriennoyl; or
R1 is hexadecanoyl and R2 is 5,8,11,14-eicosatetraenoyl; and
wherein the individual is treated, and wherein the diseases or conditions that benefits from stem cell therapy comprise myocardial injury, brain injury, spinal cord injury, muscular injury, skeletal injury, acute tubular necrosis, bowel injury, lung injury, liver injury, kidney injury, bone injury, CNS injury, Alzheimers, Parkinsons, skin injury, hernia repair, vascular anastomoses, after blunt or penetrating traumatic injury, and diabetes.

15. A method of treating an individual with a disease that benefits from stem cell therapy, the method comprises:
a. administering an amount of EC-18 to the individual, wherein the amount ranges between about 10 mg/kg and about 100 mg/kg; and
b. administering an amount of stem cells to the individual, wherein the amount of stem cells ranges between about 100,000 cells and about 160 million cells;
wherein the individual is treated.

16. The method of claim 15, wherein a disease or conditions that benefits from stem cell therapy comprises myocardial injury, brain injury, spinal cord injury, muscular injury, skeletal injury, acute tubular necrosis, bowel injury, lung injury, liver injury, kidney injury, bone injury, CNS injury, Alzheimers, Parkinsons, skin injury, hernia repair, vascular anastomoses, after blunt or penetrating traumatic injury, and diabetes.
